(19) United States
(12) Patent Application Publication
(10) Pub. No.: US 2024/0245533 A1
(43) Pub. Date: Jul. 25, 2024

(54) Knee Adapter for Connecting an Endoprosthesis to an Exoprosthesis

(71) Applicant: Back to Mobility GmbH, Hürth (DE)

(72) Inventor: Benjamin Wünsch, Hürth (DE)

(21) Appl. No.: 18/417,856

(22) Filed: Jan. 19, 2024

(30) Foreign Application Priority Data
Jan. 23, 2023 (EP) 23 152 867.0

(51) Int. Cl. A61F 2/64; A61F 2/30; A61F 2/38

(52) U.S. Cl. CPC A61F 2/64; A61F 2/3854; A61F 2002/30113

(57) ABSTRACT

A knee adapter for connecting an endoprosthesis to an exoprosthesis includes a ball joint. The ball joint comprises a partially spherical joint head and a joint socket shaped to be complementary to the joint head. The joint head is mounted in the joint socket and is configured to be arranged at one of the endoprosthesis and the exoprosthesis. The joint socket is configured to be arranged at the other of the endoprosthesis and the exoprosthesis. The joint head is affixed in the joint socket by at least one shear element.

### BACKGROUND OF THE INVENTION

### 1. Field of the Invention

The present invention relates to a knee adapter for connecting an osseointegrated implant system in the form of an endoprosthesis to an exoprosthesis. The knee adapter serves to protect the endoprosthesis from high mechanical stress and to avoid a skeletal fracture caused by an accident situation.

Those disproportionate stresses upon the endoprosthesis can arise, for example, from a stumbling motion or a fall. If such an accident situation places a disproportionate mechanical stress on, for example, the foot replacement, then this stress is passed on in the form of a strong moment directly to the connection region of the endoprosthesis installed in the bone due to the longitudinal extension of the leg prosthesis. As a result of the long lever and the resulting moment acting upon the osseointegration of the endoprosthesis, this can lead to a skeletal fracture.

### 2. Background of Related Art

Knee adapters are known from prior art, the function of which is to precisely limit these high stresses upon the endoprosthesis integrated into the bone.

US 2015/0257904 A1 discloses, among other things, a knee adapter for connecting an endoprosthesis anchored in the bone to an exoprosthesis. The knee adapter comprises a main housing having a first attachment portion arranged to attach to the endoprosthesis and a second attachment portion arranged to attach to the exoprosthesis. The knee adapter comprises in particular a safety mechanism for protecting the endoprosthesis attached to the bone from high mechanical forces, including rotational and bending forces. For this purpose, the safety mechanism comprises a mechanism for triggering when a rotation force that is too high is exceeded. Once the safety mechanism has been triggered, the exoprosthesis can be rotated about its axial extension. Furthermore, the safety mechanism comprises a bending force trigger mechanism. Once a predetermined amount of transverse force is exceeded, the bending force trigger mechanism is released and the exoprosthesis can pivot in the direction of force action.

However, the disadvantage of the knee adapter proposed in US 2015/0257904 A1 is that the safety mechanism or the bending force trigger mechanism is only triggered in the event of excessive transverse forces that act opposite to a rectilinear motion of the wearer of the prosthesis, i.e. orthogonally to the frontal plane. Lateral transverse forces, i.e. forces that act orthogonally to the sagittal plane, or transverse forces from behind, i.e. also orthogonal to the frontal plane but with an inverse orientation, do not trigger the safety mechanism. Consequently, a respective torque is passed on via the exoprosthesis and the non-triggering knee adapter to the endoprosthesis attached in the bone.

### SUMMARY

Proceeding from there, the present invention is based on the object of eliminating at least in part the disadvantages known from prior art. In addition, a user-friendly and structurally simple knee adapter is to be created.

The object is satisfied by a knee adapter for connecting an endoprosthesis to an exoprosthesis. The knee adapter comprises a ball joint. The ball joint comprises a partially spherical joint head and a joint socket shaped to be complementary thereto. The joint head is mounted in the joint socket and head is configured to be arranged at one of the endoprosthesis and the exoprosthesis. The joint socket is configured to be arranged at the other of the endoprosthesis and the exoprosthesis. The joint head is affixed in the joint socket by at least one shear element.

It is be noted as a precaution that the number words used below (“first”, “second”, . . . ) primarily serve (only) to distinguish between several similar objects, sizes or processes, i.e. that in particular no dependency and/or order of these objects, sizes or processes to each other is prescribed. Should a dependency and/or sequence be required, then this is explicitly stated there, or it will be obvious to the person skilled in the art when studying the configuration specifically described.

The knee adapter is configured to connect an osseointegrated endoprosthesis configured as an at least partial thigh replacement with an exoprosthesis that replaces the lower leg. The endoprosthesis (Greek endo “inside”) is an artificial implant that is permanently inserted into the body, at least in sections, in the context of surgery. It replaces diseased or destroyed body structures. An at least partial thigh replacement is to mean that the endoprosthesis is integrated into a remainder of the thigh bone, for example, with one end in the remaining shaft of the thigh bone, where the other end emerges from the leg stump. In addition, the knee adapter presently proposed is also suitable for an endoprosthesis that can replace the thigh bone entirely.

A prosthesis that is arranged entirely outside the body is called an exoprosthesis (Greek exo “outside”). It can be connected, for example, externally to an arm or leg stump, although a connection to an endoprosthesis is also possible, like in the present case.

The knee adapter comprises a ball joint, where the ball joint comprises a partially spherical joint head and a joint socket shaped to be complementary thereto. The joint socket and the joint head are configured relative to one another such that the joint head is mounted to be pivotable within the joint socket. In order to affix the joint head within the joint socket, at least one shear element is provided and connects the joint socket to the joint head.

The shear element is usually configured the shear off and thereby will be destroyed in case of overcritical high mechanical forces, including rotational and bending forces, transmitted via the joint. This instance brings about the need to put a new shear element in place. Mechanical properties and dimensions of the shear element can be altered to set the value of overcritical high mechanical forces resulting into failure of the shear element.

A further shear element may provided, where the shear element and the further shear element are aligned orthogonally to one another. The shear element and the further shear element are each arranged with one end in the joint socket and with the other end in the joint head. Furthermore, the shear elements can also be aligned in different angular positions relative to one another, where further shear elements can particularly preferably also be provided.

The shear element and the further shear element are provided in order to enable optimal fixation of the joint head within the joint socket, in other words to block any motion of the joint head within the joint socket. The two shear elements are each arranged with one end in the joint socket and with the other end in the joint head. The shear element blocks a translational and rotatory motion of the joint head within the joint socket in a frontal plane and in a sagittal plane, whereas the further shear element blocks a translational and rotatory motion of the joint head within the joint socket in a transverse plane and in the frontal plane. The first shear element generally blocks all rotations except for one rotation about the proximal/lateral axis, the second element generally blocks all rotations except for one rotation about the medial/lateral axis. In principle, however, an inverse or completely different arrangement of the shear elements is also preferred as an alternative.

In anatomy, the frontal plane is a plane that extends from top to bottom and from left to right when a person is standing upright. A frontal plane divides a body into a front and a back portion. In anatomy, the sagittal plane is a plane that extends from top to bottom and from back to front. A sagittal plane divides a body into right and left portions. In anatomy, a transverse plane is a plane that is disposed transverse at a right angle to the longitudinal axis. It stretches horizontally when the longitudinal axis is vertical and extends from front to back and from left to right when a person is standing upright. Such a transverse plane is also referred to as a horizontal plane and divides a body into an upper and a lower portion.

The shear elements are each configured with a fracture defining segment. Once a certain amount of force or moment acting upon them is exceeded, they fracture and release the joint head within the joint socket. The joint head can then pivot freely within the joint socket, possibly up to a lateral stop of the joint socket, without transmitting any force to the connection between the endoprosthesis and the bone. In addition, the exoprosthesis is typically also released relative to the endoprosthesis in a rotatory direction. In other words, the exoprosthesis can rotate relative to the endoprosthesis. possibly also in the axial direction.

The advantage of the ball joint is that, after the shear elements fracture, the exoprosthesis can avoid transverse forces or torsional stresses in any direction and pivots in their direction of action. Therefore, only a safety mechanism in the form of the ball joint is necessary to provide protection against excessive mechanical stresses that could act upon the exoprosthesis from 360° in the axial direction.

The shear element may engage in the joint socket, starting out from the inner side of the joint head, where the further shear element engages in the joint head from the outer side of the joint socket. The arrangement of the shear element in the joint head enables a compact design of the knee adapter. However, an inverse arrangement of the respective shear element is also possible.

The shear element and the further shear element may be formed from stainless steel, where the shear element is sized to be larger than the further shear element and has a higher fracture strength against shearing stress. The shear elements are each formed from brittle stainless steel and have low ductility. Low ductility has the advantage that, under a precisely defined stress caused by a moment or a force that acts as shearing forces upon the shear element, the shear element fractures, i.e. gives way without any previous plastic deformation. Since experience and evaluations show that the highest stresses act upon the exoprosthesis in the direction and opposite to the direction of locomotion, i.e. orthogonally to the frontal plane, the shear element is sized to be stronger than the further shear element. However, stresses transverse to the direction of locomotion, i.e. orthogonally to the sagittal plane, of the user are weaker, which is why the further shear element is sized to be weaker.

The joint head may comprise a bridge module on the side facing away from the joint socket, where the bridge module is configured to be complementary to a connection region of the endoprosthesis and/or the exoprosthesis. The endoprosthesis or exoprosthesis is in turn provided with a connection region which is configured to be complementary to the bridge module and which can be received in the bridge module. The bridge module may be configured as a hollow cylinder and is connected integrally to the joint head. The end of the endoprosthesis or exoprosthesis on the bridge module side, however, is configured as a cylinder that is complementary to the inner side of the hollow cylinder and can be inserted into the hollow cylinder. However, other complementary forms of connection between the bridge module and the endoprosthesis are also conceivable and sometimes advantageous. The hollow cylinder may also taper or the cylindrical section of the endoprosthesis can expand slightly in the direction away from the bridge module, respectively, in order to produce a press fit during insertion.

For a force-fit connection of the endoprosthesis or exoprosthesis to the bridge module, an attachment element may be arranged at the circumference of the side of the bridge module facing away from the joint socket. The attachment element can be any device that creates a force-fit and/or frictionally engaged and/or positive-ft connection between the bridge module and the endoprosthesis or exoprosthesis. To make it easier for the user to use, the attachment element may be configured, for example, as an annular quick-release tensioner circumferentially around the region of the bridge module. A force-fit connection is therefore caused by tensioning a lever. A positive-fit connection between the endoprosthesis and the bridge module by way of the attachment element is also possible.

The joint head may comprise an equator on a plane of a normal to its axial extension, where the joint socket surrounds the joint head at least in part beyond this equator in the direction of the bridge module. This nesting of the joint head, when viewing the knee adapter in the axial direction beyond the 90°, prevents the joint head from falling out of the joint socket. In other words, the socket surface of the joint socket is formed to be more than hemispherical such that its radius in the opening region has already reduced compared to the radius of the equator.

A region of the joint socket that radially surrounds the joint head may be divided into at least two sections, where the sections are divided by recesses in the axial extension of the joint socket, starting out from the side of the joint socket facing the bridge module. The sections may be formed to be resilient so that the joint head can be levered out of the joint socket. In addition, the division of the joint socket into several regions leads to better deformability of the joint socket when the joint head is levered out as well reinserted. Furthermore, the resilient regions prevent the joint socket from fracturing when the joint head is levered out. The resiliency of the sections is selected such that a certain moment or a certain force leads to a levering out action, where this moment or this force is uncritical for the connection region between the endoprosthesis and the thigh bone.

Levering the joint head out of the joint socket and thereby separating the exoprosthesis from the endoprosthesis is advantageous for the reason that, in the event of an accident, despite pivoting the exoprosthesis relative to the endoprosthesis after the shear elements have fractured, excessive mechanical stress would act upon the connection region between the endoprosthesis and the bone once the joint head has reached its maximum pivoting within the joint socket.

Th Each of the sections may at an end facing the bridge module, abut with at least one projection against the joint head. The projections ensure that the divided and resilient regions of the joint socket abut against the surface of the joint head under tension. A play-free fit of the joint head in the joint socket is thus achieved. The projections also ensure easier assembly of the joint head within the joint socket, as they take on the function of locking elements.

### BRIEF DESCRIPTION OF THE DRAWINGS

The invention and the technical environment shall be explained in more detail below using the figures. It is be noted that the invention is not intended to be restricted by the embodiments shown. Unless explicitly stated otherwise, it is also possible, in particular, to extract partial aspects of the circumstances explained in the figures and to combine them with other components and findings from the present description and/or figures. It is to be noted in particular that the figures and in particular the proportions shown are only schematic. The same reference characters designate the same objects so that explanations from other figures can additionally be used where necessary, where:

FIG. 1 shows a schematic sectional view of a knee adapter according to the invention, arranged at an endoprosthesis and exoprosthesis;

FIG. 2 shows a schematic representation of the underside of the knee adapter according to the invention;

FIG. 3 shows a schematic sectional view of the knee adapter according to the invention with overstressed shear elements;

FIG. 4 shows a schematic sectional view of the knee adapter according to the invention with the joint head disengaged from the joint socket and

FIG. 5 shows a schematic side view of the knee adapter according to the invention.

### DETAILED DESCRIPTION

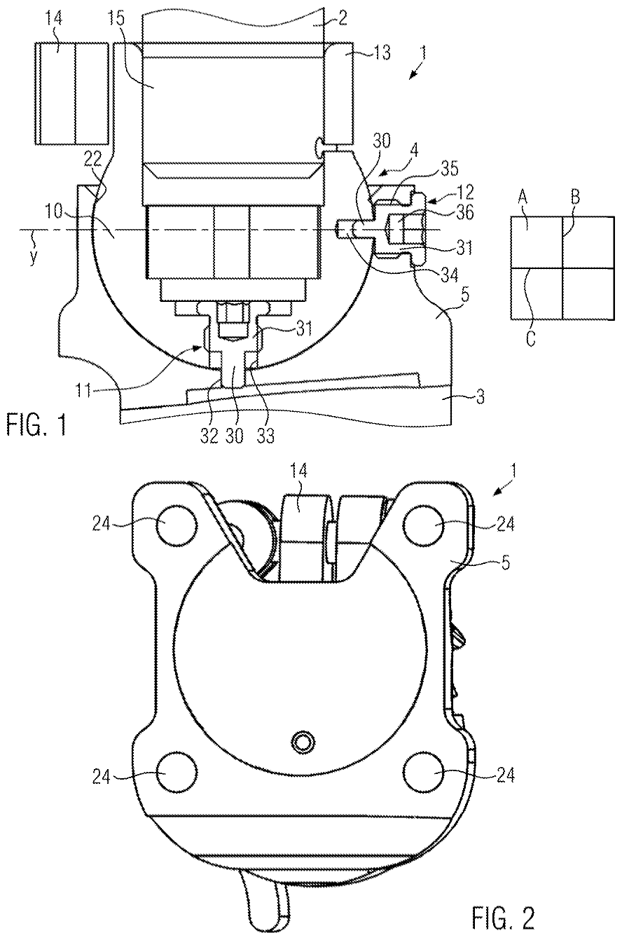

FIG. 1 shows a schematic representation of a knee adapter 1 according to the invention. The knee adapter 1 is configured to connect an osseointegrated endoprosthesis 2 configured as an at least partial thigh replacement to an exoprosthesis 3 replacing the lower leg. The knee adapter 1 comprises a ball joint 4, where the ball joint 4 comprises a partially spherical joint head 10 and a joint socket 5 shaped to be complementary thereto. The joint socket 5 and the joint head 10 are configured relative to one another such that the joint head 10 is mounted to be pivotable within the joint socket 5. The joint head 10 comprises an equator Y on a plane of the normal to its axial extension, where the joint socket 5 surrounds the joint head 10 at least in part beyond this equator Y in the direction of the bridge module 13. This nesting of the joint head 10, when viewing the knee adapter 1 in the axial direction beyond the 90°, prevents the joint head 10 from falling out of the joint socket 5.

The joint head 10 is formed with a hollow cylindrical bridge module 13 on the side facing away from the joint socket 5 for connecting the endoprosthesis 2 to the exoprosthesis 3. The endoprosthesis 2 is in turn provided with a connection region 15 which is configured to be complementary to the hollow cylindrical bridge module 13 and has the shape of a cylindrical section 15 which is received in the bridge module 13. In order to securely connect the cylindrical section 15 of the endoprosthesis 2 in the hollow cylindrical bridge module 13, the bridge module 13 is provided with an attachment element 14. The attachment element 14 is formed as an annular quick release tensioner circumferentially around the region of the bridge module 13. In order to create a connection between the endoprosthesis 2 and the bridge module 13, the bridge module 13 comprises a joint gap 23 which enables the bridge module 13 to be compressed by the attachment element 14, thus establishing a self-locking frictional connection, i.e. force-fit connection between the endoprosthesis 2 and the bridge module 13.

In addition, the joint socket 5 on the side facing away from the joint head 10 has a flange-shaped structure which is configured to establish a connection with the exoprosthesis 3. In order to establish a force-fit connection of the endoprosthesis 2 to the flange-shaped structure of the joint socket 5, the flange-shaped structure of the joint socket 5, as shown in FIG. 2, comprises four threaded bore holes 24 which are configured to receive nuts, not shown, that connect the exoprosthesis 3 to the flange- shaped structure of the joint socket 5 in a force-fit and positive-fit manner to each other.

In order to affix the joint head 10 within the joint socket 5, a shear element 11 and a further shear element 12 which block a motion of the joint head 10 within the joint socket 5 are provided. For this purpose, the two shear elements 11, 12 are each arranged with one end in the joint socket 5 and with the respective other end in the joint head 10. The shear elements 11, 12 are arranged orthogonally in the axial direction to one another, where the shear element 11 blocks a rotatory motion of the joint head 10 within the joint socket 5 in frontal plane A and in sagittal plane B, whereas the further shear element 12 blocks a rotatory motion of the joint head 10 within the joint socket 5 in transverse plane C and in frontal plane A.

The shear elements 11, 12 can each be divided into two sections 30, 31, namely into a threaded section 31 and a coaxially subsequent radially tapered cylindrical section 30. Starting out from the inner side of the joint head 10, the shear element 11 engages with its tapered cylindrical section 30 into a first bore hole 32 within the joint socket 5, where the threaded section 31 is screwed into a first threaded bore hole 33 in the joint head 10. In other words, the shear element 11 is screwed from within the joint head 10 into the first threaded bore hole 33. The first threaded bore hole 33 is formed to be coaxial with the hollow cylindrical bridge module 13 and extends parallel to the intersection line between the frontal plane A and the sagittal plane B.

Starting out from the outer side of the joint socket 5, the further shear element 12, on the other hand, engages with its tapered cylindrical section 30 into a second bore hole 34 within the joint head 10, where the threaded section 31 is screwed into a second threaded bore hole 35 in the joint socket 5. In other words, the further shear element 12 is screwed into the second threaded bore hole 35 from outside the joint socket 5 and is therefore accessible from the outside. The second threaded bore hole 35 is formed to be orthogonal to the first threaded bore hole 33 and extends parallel to the intersection line between the sagittal plane B and the transverse plane C.

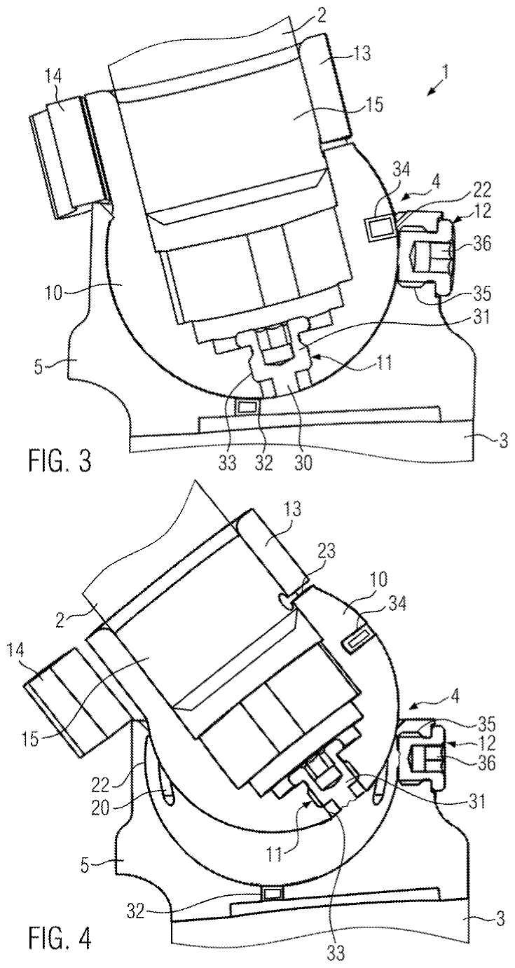

According to FIGS. 3 and 4, the cylindrical sections 30 of the shear element 11 and the further shear element 12 are configured with fracture defining segments. Once a certain amount of force or moment acting upon them is exceeded, the cylindrical sections 30 fracture and release the joint head 10 within the joint socket 5. The joint head 10 can then pivot freely within the joint socket 5, as is shown in FIG. 3. In order to enable the shear elements 11, 12 to be replaced after an overstress and the associated fracture of the cylindrical section 30 from the respective threaded section 31, the respective threaded section 31 is provided with a blind bore hole 36, where the blind bore holes 36 are configured to receive a hexagonal key. As already explained, the further shear element 12 can be replaced in a simple manner from outside the ball joint 4 after fracture. In order to replace the shear element 11 after fracture, the endoprosthesis 2 must first be separated from the knee adapter 1 and from the bridge module 13, respectively, whereupon the shear element 11 can be replaced from within the joint head 10.

The shear elements 11, 12 are each formed from stainless steel and have low ductility. Low ductility has the advantage that, under a precisely defined stress caused by a moment or a force, the cylindrical section(s) 30 fracture immediately, i.e. without any prior plastic deformation. Alternatively, the shear elements 11, 12 can also be formed from a different material with low ductility, for example, ceramic material. Since, statistically speaking, the highest stresses act in the direction and opposite to the direction of locomotion of the prosthesis wearer upon the exoprosthesis 3 orthogonally, i.e. in the direction of the frontal plane A, the shear element 11, more precisely the cylindrical section 30, is sized to be stronger than the further shear element 12. The shear element 11 therefore has a higher fracture strength against shearing stress than the further shear element 12. However, stresses transverse to the direction of locomotion of the user, i.e. in the direction of the sagittal plane B, are weaker, for which reason the further shear element 12 is sized to be weaker. Its stress is also less during normal operation so that the mechanical stress threshold for the fracture of the further shear element 12 can be structurally reduced.

Furthermore, the pure torsional stress is much less than the bending stress in the event of a fall or the like. Therefore, the shear element 12 is sized to be smaller than the shear element 11, since only the shear element 12 shears under a pure torsional stress. In the event of a bending stress, either both shear elements will shear off (large stress) or, in the event of pure bending around the frontal/transverse sectional axis, only the shear element 11, depending on the axis.

Since the two shear elements 11, 12 are oriented differently relative to one another, it is also possible that only one of the shear elements 11, 12 fractures when a corresponding mechanical stress threshold is exceeded. A pivoting motion of the joint head 10 relative to the joint socket 5 in the axial direction around the shear element 11, 12 that still remains would then be possible.

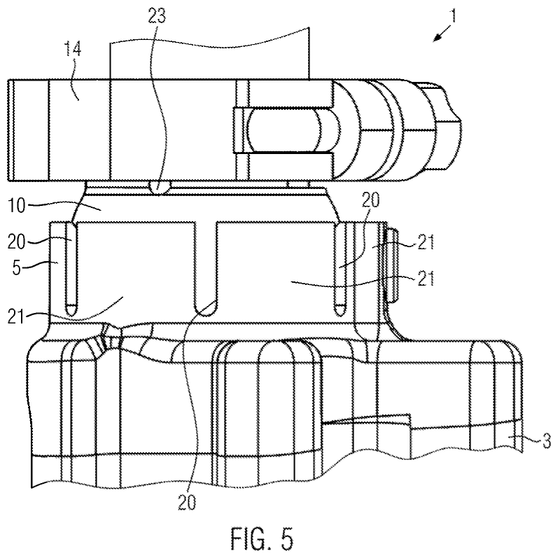

As shown in FIG. 4, the knee adapter 1 is configured such that the joint head 10 can be levered out of the joint socket 5 after the shear elements 11, 12 have fractured. Should a force or moment continue to act upon the exoprosthesis 3, even after the shear elements 11, 12 have fractured, and thereby a potential fracture of the connection region between the remainder of the thigh bone and the endoprosthesis 2 exist, then the exoprosthesis 3 can become separated from the endoprosthesis 2. For this purpose, a region of the joint socket 5 that radially surrounds the joint head 10 is divided into several sections 21 (FIG. 5). The sections 21 are divided by recesses 20 in the axial extension of the joint socket 5, starting out from the side of the joint socket 5 facing the bridge module 13. In order to enable the joint head 10 to be levered out of the joint socket 5, the sections 21 are configured to be elastic. This elasticity results primarily from the shape of the sections 21. In addition, the division of the joint socket 5 into several sections 21 leads to better deformability of the sections 21 of the joint socket 5 when the joint head 10 is levered out as well as reinserted into the joint socket 5. Furthermore, the resilient sections 21 prevent the joint socket 5 from fracturing when the joint head 10 is levered out.

The resilience of the sections 21 is selected such that a certain moment or a certain force leads to a levering out action, where this moment or the force is uncritical for the connection region between the endoprosthesis 2 and the thigh bone. In order to be able to structurally better implement this force or torque threshold, the sections 21 each at their end facing the bridge module 13 abut with a projection 22 against the joint head 10. This also ensures facilitated assembly of the joint head 10 within the joint socket 5. The projections 22 also ensure that the divided and resilient sections 21 of the joint socket 5 abut against the surface of the joint head 10 under tension. A play-free fit of the joint head 10 in the joint socket 5 is thus achieved.

1. A knee adapter for connecting an endoprosthesis to an exoprosthesis, the knee adapter comprising:
a ball joint, wherein said ball joint comprises a partially spherical joint head and a joint socket shaped to be complementary thereto, wherein said joint head is mounted in said joint socket, wherein said joint head is configured to be arranged at one of said endoprosthesis and said exoprosthesis, wherein said joint socket is configured to be arranged at the other of said endoprosthesis and said exoprosthesis, wherein the joint head is affixed in said joint socket by at least one shear element.

2. The knee adapter according to claim 1, further comprising a further shear element, wherein said shear element and said further shear element are aligned orthogonally to one another, wherein said shear element and said further shear element are each arranged with one end in said joint socket and with another other end in said joint head.

3. The knee adapter according to claim 1, wherein, starting out from an inner side of said joint head, said shear element engages in said joint socket.

4. The knee adapter according to claim 2, wherein said shear element and said further shear element are formed from stainless steel, and wherein said shear element has a higher fracture strength against shearing stress than said further shear element

5. The knee adapter according to claim 1, wherein said joint head comprises a bridge module on a side facing away from said joint socket, wherein said bridge module is configured to be complementary to a connection region of said endoprosthesis and/or said exoprosthesis.

6. The knee adapter according to claim 5, wherein an attachment element is arranged at a circumference on a side of said bridge module facing away from said joint socket.

7. The knee adapter according to claim 5, wherein said joint head comprises an equator (Y) on a plane of the normal to an axial extension thereof, and wherein said joint socket surrounds said joint head, at least in part, beyond this equator (Y) in the direction of said bridge module.

8. The knee adapter according to claim 5, wherein a region of said joint socket that radially surrounds said joint head is divided into at least two sections, and wherein said sections are divided by recesses in an axial extension of said joint socket, starting out from the side of said joint socket facing said bridge module.

9. The knee adapter according to claim 8, wherein each of said sections has an end that faces said bridge module and that abuts with at least one projection against said joint head.

10. The knee adapter according to claim 8, wherein said sections are configured to be resilient.

11. A knee adapter for connecting an endoprosthesis to an exoprosthesis, the knee adapter comprising:
a ball joint, wherein said ball joint comprises a partially spherical joint head and a joint socket shaped to be complementary thereto, wherein said joint head is mounted in said joint socket, wherein said joint head is configured to be arranged at one of said endoprosthesis and said exoprosthesis, wherein said joint socket is configured to be arranged at the other of said endoprosthesis and said exoprosthesis, wherein said joint head is affixed in said joint socket by at least one shear element such that, when a certain amount of force or moment acting upon said shear element is exceeded, a predetermined fracture defining segment of said shear element fractures and releases said joint head within said joint socket.

12. The knee adapter according to claim 11, further comprising a further shear element, wherein said shear element and said further shear element are aligned orthogonally to one another, wherein said shear element and said further shear element are each arranged with one end in said joint socket and with another end in said joint head.

13. The knee adapter according to claim 11, wherein, starting out from an inner side of said joint head, said shear element engages in said joint socket.

14. The knee adapter according to claim 12, wherein said shear element and said further shear element are formed from stainless steel, wherein said shear element has a higher fracture strength against shearing stress than said further shear element

15. The knee adapter according to claim 11, wherein said joint head comprises a bridge module on a side facing away from said joint socket, wherein said bridge module is configured to be complementary to a connection region of said endoprosthesis and/or said exoprosthesis.

16. The knee adapter according to claim 15, wherein an attachment element is arranged at a circumference on a side of said bridge module facing away from said joint socket.

17. The knee adapter according to claim 15, wherein said joint head comprises an equator (Y) on a plane of the normal to an axial extension thereof, wherein said joint socket surrounds said joint head, at least in part, beyond this equator (Y) in the direction of said bridge module.

18. The knee adapter according to claim 15, wherein a region of said joint socket that radially surrounds said joint head is divided into at least two sections, and wherein said sections are divided by recesses in an axial extension of said joint socket, starting out from a side of said joint socket facing said bridge module.

19. The knee adapter according to claim 18, wherein each of said sections has an end that faces said bridge module and that abuts with at least one projection against said joint head.

20. The knee adapter according to claim 18, wherein said sections are configured to be resilient.
